SEQUENCE LISTING (22 sequences)

SEQ ID NO: 1 - protein, 5 aa, Artificial Sequence
  Sequence Motif
LXCXE

SEQ ID NO: 2 - protein, 5 aa, Artificial Sequence
  Sequence Motif
LXCXD

SEQ ID NO: 3 - protein, 22 aa, Human cytomegalovirus
SGDGYHGLRCRETSAMWSFEYD

SEQ ID NO: 4 - protein, 22 aa, Chimpanzee cytomegalovirus
GGSSYHGLRCRESSDMWSFEYE

SEQ ID NO: 5 - protein, 20 aa, Simian virus 40
NAFNEENLFCSEEMPSSDDE

SEQ ID NO: 6 - protein, 19 aa, Human adenovirus 52
RGEELDKCYEECLPSSDSE

SEQ ID NO: 7 - protein, 20 aa, Human papillomavirus
QPETTDLYCYEQLNDSSEEE

SEQ ID NO: 8 - protein, 33 aa, Human herpesvirus 6A
DCSAIEELLCHESLLDSPMKLSNAHTMFSSDKW

SEQ ID NO: 9 - protein, 33 aa, Human herpesvirus 6B
DCSAIEELLCHESLLDSPMKLSNAHTIFSSNKW

SEQ ID NO: 10 - protein, 33 aa, Human herpesvirus 7
PDPSVTDLLCHESLTVSPKFERDGLSACTEFEN

SEQ ID NO: 11 - protein, 33 aa, Human cytomegalovirus
LVDALEQLACSDPNTYIHKTETDERGQWIMLFL

SEQ ID NO: 12 - protein, 32 aa, Human cytomegalovirus
HHDQWKLACIDSYRRAFCTLADAIKFLNHQCR

SEQ ID NO: 13 - protein, 4 aa, Artificial Sequence
  DSSE Motif
DSSE

SEQ ID NO: 14 - DNA, 25 bp, Artificial Sequence
  Primer
caccatgtcctccgcacttcggtct

SEQ ID NO: 15 - DNA, 29 bp, Artificial Sequence
  Primer
tcaccttcgaccgcccgtagctgtcgatg

SEQ ID NO: 16 - DNA, 19 bp, Artificial Sequence
  Primer
ttactcggggaacagttgg

SEQ ID NO: 17 - DNA, 29 bp, Artificial Sequence
  Primer
agtggaagctggcgggcatcgacagctac

SEQ ID NO: 18 - DNA, 27 bp, Artificial Sequence
  Primer
ggtgccgaacgcgccggcgctttgaag

SEQ ID NO: 19 - DNA, 27 bp, Artificial Sequence
  Primer
ccacggcttgcgcggccgcgaaacttc

SEQ ID NO: 20 - protein, 33 aa, Artificial Sequence
  Consensus Sequence
XXDAXXDLXCHESXXXXXXXXXXXXXMWSSDXE

SEQ ID NO: 21 - DNA, 80 bp, Artificial Sequence
  Primer
ggccttacgtgcgacccgcgtatgttcttgcgccttacgcatcccgagctctgcgacctgttgacaattaatcatcggca

SEQ ID NO: 22 - DNA, 80 bp, Artificial Sequence
  Primer
atcttgtggcaaaaatcgtcctctttgggcacgtagaccagcaggtaggagatagagagctcagcactgtcctgctcctt

(19) United States
(12) Patent Application Publication
(10) Pub. No.: US 2010/0143440 A1
(43) Pub. Date: Jun. 10, 2010

(54) UL97 INHIBITORS FOR TREATMENT OF PROLIFERATIVE DISORDERS

(75) Inventor: Mark N. Prichard, Birmingham, AL (US)

Correspondence Address: FISH & RICHARDSON P.C., P.O BOX 1022, Minneapolis, MN, 55440-1022, US

(73) Assignee: The UAB Research Foundation, Birmingham, AL (US)

(21) Appl. No.: 12/598,389

(22) PCT Filed: Mar. 4, 2008
(86) PCT No.: PCT/US08/55802

§ 371 Date: Dec. 22, 2009

Related U.S. Application Data
(60) Provisional application No. 60/914,901, filed on Apr. 30, 2007.

(51) Int. Cl. A61K 9/00; A61K 31/517; A61K 31/553; A61K 31/706; A61K 31/522; A61K 31/35; A61K 31/55; A61K 31/7056; A61K 31/445; A61K 31/713; A61K 31/7105; A61K 38/08; A61K 39/395; A61K 45/00; G01N 33/53; A61P 31/12; A61P 37/04

(52) U.S. Cl. 424/423; 514/266.1; 514/211.08; 514/27; 514/263.38; 514/453; 514/218; 514/43; 514/320; 514/44 R; 514/44 A; 514/16; 424/130.1; 424/93.2; 435/7.2

(57) ABSTRACT

Methods and compositions related to treating or preventing a proliferative disease in a subject comprising administering an inhibitor of a UL97 or a UL97 homolog to the subject are described.

### CROSS-REFERENCE TO PRIORITY APPLICATIONS

This application claims priority to U.S. Provisional Application No. 60/914,901, filed Apr. 30, 2007, which is incorporated herein by reference in its entirety.

### STATEMENT REGARDING FEDERALLY SPONSORED RESEARCH

This invention was made with government support under Grant No. NOI-AI-30049 awarded by the National Institutes of Health. The government has certain rights in the invention.

### BACKGROUND

The Herpesviridae is a large family of DNA viruses that cause diseases in humans and animals. There are eight distinct viruses in this family known to cause disease in humans: HHV-1, HHV-2, HHV-3, HHV-4, HHV-5, HHV-6, HHV-7 and HHV-8. Latent, re-occurring or lytic infections are typically associated with this group of viruses. Common infections include: herpes, CMV, chickenpox, shingles, mononucleosis, measles, and Kaposi sarcoma. The herpesviruses may live latently in a person for years or even decades without causing symptoms and then be activated and cause disease.

HHV are associated with a number of diseases including cancer, organ rejection, graft versus host disease and heart disease. There are anti-viral agents available to treat herpesvirus infections including idoxuridine, trifluridine, vidarabine and acyclovir for the topical treatment of herpetic infections; vidarabine and acyclovir for the systemic (intravenous) treatment of herpes encephalitis; acyclovir for the topical and systemic (oral) treatment of genital herpes; acyclovir for the systemic (intravenous, oral) treatment of HSV or varicella-zoster (VZV) infections in immunosuppressed patients; brivudin for the systemic (oral) treatment of HSV-1 or VZV infections in immunosuppressed patients; and ganciclovir and foscarnet for the systemic (intravenous) treatment of cytomegalovirus (CMV) retinitis in AIDS patients. Other antiviral agents considered for use in herpesvirus infections include brovavir, penciclovir (and its prodrug famciclovir), desciclovir (a prodrug of acyclovir), bishydroxymethylcyclobutylguanine (BHCG) and, in particular, 1-(3-hydroxy-2-phosphonylmethoxypropyl)cytosine (HPMPC). However, development of drug resistance is still a common problem and there is a lack of sufficient and effective treatments for diseases associated with herpesviruses.

### SUMMARY

Provided is a method of treating or preventing a proliferative disease in a subject comprising selecting a subject with a proliferative disease and administering an inhibitor of a UL97 or a UL97 homolog to the subject. Inhibitors of a UL97 or a UL97 homolog include inhibitory peptides, drugs, functional nucleic acids and antibodies. Also provided are medical devices comprising an inhibitor of a UL97 or a UL97 homolog.

The details of one or more embodiments are set forth in the accompanying drawings and the description below. Other features, objects, and advantages will be apparent from the description and drawings, and from the claims.

### DESCRIPTION OF DRAWINGS

FIG. 1 shows IE1 and pp71 promote the formation of pp65-GFP nuclear aggregates. Cells co-expressing the pp65-GFP fusion protein with the other viral proteins shown were counted and the percentage containing nuclear aggregates was calculated. The value shown is the average of 10 fields with the standard deviation represented by the error bars.

FIGS. 2 A-U show inhibition of aggregate formation by UL97 kinase. Aggregates induced through the expression of pp65, pp71 and GFP-GCP 170* are shown in panels A-C, respectively. Aggregate formation is inhibited in cells that co-express pp65-GFP and UL97 (panels D-F). The kinase negative mutant (K355M) is unable to inhibit aggregation of pp65-GFP and is recruited to the aggregates (panels G-I). The kinase also inhibits the formation of pp71 aggregates in co-transfected cells (panels J-L). The K355M mutant is unable to affect pp71 aggregates and is recruited to the aggregates (panels M-O). Aggresomes induced through the expression of the cellular protein, GFP-GCP170* are inhibited by the kinase (panels P-R). Aggregation of GFP-GCP170* is unaffected by the K355M mutant, which is specifically recruited to cytoplasmic aggresomes (panels S-U).

FIGS. 3 A-F show that UL97 kinase reduces the number of PML domains in COS7 cells. Cells were transfected with (A) ppUL44, (B) IE1, (C) pp71, (D) UL97, (E) K355M, and (F) UL97 with the addition of MBV. Expression of pUL97 reduced the number of PML bodies in a kinase dependent manner. Values shown in each panel are average number of PML domains per cell and reflect the average of at least 50 cells with the standard error of the mean shown.

FIG. 4 shows that UL97 kinase activity is required for the hyperphosphorylation of Rb in infected cells. Cell lysates from mock infected cells, cells infected with AD169, or cells infected with a UL97 deleted virus (UL97Δ) were separated on polyacrylamide gels and transferred to PVDF membranes. Samples were obtained at 24 and 72 hours following infection and half of the infections occurred in the presence of MBV, as indicated in the figure. Shown are immunoblots using monoclonal antibodies to the proteins shown to the left of the figure. Hyperphosphorylated forms of the retinoblastoma protein (Rb) are reduced when the kinase is deleted or when its activity is inhibited with maribavir (MBV).

FIG. 5A shows Rb binding motifs in pUL97. Amino acid sequences of viral proteins containing LxCxE (SEQ ID NO:1) and LxCxD (SEQ ID NO:2) motifs were aligned with the motifs identified in pUL97. Sequences for HCMV LxCxE (NP_{—}040032.1) (SEQ ID NO:3), CCMV UL97 (NP_{—}612729) (SEQ ID NO:4), SV40 large T (NP_{—}043127) (SEQ ID NO:5), HAdV E1A (ABK35030.1) (SEQ ID NO:6), HPV 16 E7, (AAD33253.1) (SEQ ID NO:7), HHV-6A U69 (NP_{—}042962.1) (SEQ ID NO:8), HHV-6B U69 (T44214) (SEQ ID NO:9), HHV-7 U69 (YP_{—}073809.1) (SEQ ID NO:10), HCMV pp71 (NP_{—}040017) (SEQ ID NO:11), and HCMV LxCxD (NP_{—}040032.1) (SEQ ID NO:12), are shown with the consensus sequence (SEQ ID NO:20).

FIG. 5B shows epitope tagged versions of pUL97 expressed in COS7 cells and immunoprecipitated with a monoclonal antibody to the V5 epitope tag. The K355M mutation eliminates kinase activity, whereas C428G and C141G disrupt the LxCxD (SEQ ID NO:2) and LxCxE (SEQ ID NO:1) motifs, respectively. UL89 and UL104 fusion proteins serve as negative controls and Rb alone was used as a positive control. Immunoblot shown in the top panel shows the expression of the immunoprecipitated fusion proteins. The bottom blot shows the Rb bound to the precipitated UL97 proteins but not to the negative controls.

FIG. 6 shows the LxCxE (SEQ ID NO:1) motif in the kinase is required for the inhibition of aggregate formation. Cells co-expressing pp65-GFP with the proteins shown were counted and the percentage of cells that exhibited nuclear aggregates were calculated. The values shown are the average of three separate experiments with the standard deviations shown.

FIG. 7 shows a schematic model of UL97 kinase function in aggregate formation.

FIG. 8 shows HHV-6B U69 protein kinase is inhibited by MBV. Cells were transfected with constructs expressing UL27, UL97, UL69 or UL69B K219M and treated with 15 μM MBV (dark gray bars) or untreated (light gray bars). Aggregation of pp65-GFP is inhibited by both the HCMV UL97 kinase and the HHV-6 U69 kinase (light gray bars). Inhibition of aggregation is kinase dependent since the U69B K219M mutation, which eliminates an essential lysine, is incapable of inhibiting aggregation. Treatment of infected cells with 15 μM MBV reduces the ability of UL97 and U69 to inhibit aggregation (dark gray bars).

FIG. 9 shows recombinant viruses with point mutations in either the LxCxE RB binding motif or the kinase motif were impaired in their ability to stabilize and phosphorylate RB. HFF cells were infected at an MOI of 2 PFU/cell with the wild-type virus HB5, or with recombinant viruses containing point mutations in pUL97 as shown. Cell lysates were harvested at 24 hours following infection, separated on polyacrylamide gels and transferred to nitrocellulose membranes. Shown are immunoblots using antibodies to the proteins indicated in the left margin of the figure. Phosphorylation of RB on serine 780 was determined with specific antisera. Expression of IE1 confirmed that cells were infected and tubulin was included as a loading control.

### DETAILED DESCRIPTION

Each of the human herpesviruses encode at least one well conserved serine/threonine protein kinase that are important in viral infection and may phosphorylate substrates that are also targets of cdc2. Herpes simplex virus (HSV) UL13 and Epstein-Barr virus (EBV) BGLF4 phosphorylate eukaryotic elongation factor 1delta, and HSV UL13 and human cytomegalovirus (HCMV) pUL97 both phosphorylate the carboxyl-terminal domain of RNA polymerase II. Many other activities on cellular proteins have been described, such as the activation of cdc2 by HSV UL13, the inhibition of histone acetylation and activation of protein kinase A by HSV UL3. Viral proteins are also substrates of these kinases and the DNA polymerase processivity factors of these viruses also appear to be substrates.

The UL97 protein kinase in HCMV is particularly important because of its relevance to antiviral therapy. This enzyme phosphorylates and thus activates the antiviral drug ganciclovir (GCV), which is the treatment of choice for these infections. It is also the molecular target of the maribavir (MBV), which is a potent and selective inhibitor of its enzymatic activity, has good antiviral activity both in vitro and in vivo, and is well tolerated in human subjects. The kinase is a tegument protein expressed with early/late kinetics, which autophosphorylates amino terminal serine and threonine. A recombinant virus with a large deletion in UL97 replicates poorly in its absence and virus titers are reduced more than 100-fold suggesting that it is critical in the replication of the virus. Many defects have been described in nuclei of cells infected with the null mutant and include modestly reduced DNA synthesis, inefficient DNA packaging, and the failure of mature capsids to reach the cytoplasm.

Viruses have evolved diverse and elegant strategies to create a favorable replication environment by usurping and manipulating host cellular factors to replicate their own genome. SV40 LTag, HPV E6 and E7 proteins, and Ad E1A and E1B proteins are capable of subverting for viral replication the otherwise tightly controlled host cell cycle. The p53 and retinoblastoma protein (Rb) play key roles in controlling progression through the cell cycle, whereby p53 exerts its effect on the G2-M and G1-S transition and Rb exerts its effect on the G1-S transition. Inactivation of p53 or Rb function results in uncontrolled cell division. The Rb transcriptional repressor is a member of the pocket protein family, which also includes p130 and p107, and binds and represses transcriptional activation by the E2F/DP family of DNA-binding proteins. Sequential phosphorylation by cyclin-dependent kinases at the end of the G1 phase leads to dissociation of Rb/E2F/DP complexes, which in turn activates the expression of cellular factors required for S-phase entry. Viruses have developed an efficient way to inactivate the Rb checkpoint, by stimulating the disassembly of the Rb/E2F/DP complex during the G1-S cell cycle transition, leading to the production of host enzymes required for replication of the virus genome. The SV40 LTag, HPV E7, and Ad E1A viral oncoproteins employ a strictly conserved LxCxE motif to mediate high-affinity binding to Rb.

Described herein is UL97, the viral kinase from CMV, required for the inactivation of Rb. As described in the examples below, the kinase is required for the hyperphosphorylation of Rb. Two consensus Rb binding motifs, one of which was conserved in all the human β-herpesviruses, are identified. Specifically, the Rb binding motif LxCxE (SEQ ID NO:1) was conserved in all viral kinases of human β-herpesviruses, CMV UL97, HHV-6A and 6B U69 and HHV-7 U69. The DSSE (SEQ ID NO:13) motif is also conserved among CMV UL97, HHV-6A and 6B U69. The other consensus Rb binding motif is LxCxD (SEQ ID NO:2), which resembles the LxCxD binding motif of pp71. Pp71 is also involved in the modification of Rb.

Provided herein is a method of treating or preventing a proliferative disease in a subject comprising selecting a subject with a proliferative disease and administering an inhibitor of a UL97 to the subject.

As used herein, the term proliferative disease is a disease associated with a herpes virus. Preferably, the herpes virus is CMV, EBV, HHV-6A, HHV-6B or HHV-7.

Proliferative diseases associated with herpes viruses include, but are not limited to, heart disease, restenosis, inflammatory disease, lymphoproliferative disorders, multiple sclerosis, organ rejection, Kaposi's sarcoma, transplant arteriosclerosis, myocarditis, retinitis, obliterative bronchiolitis and neoplastic disorders. Optimally the proliferative disease is not graft versus host disease (GvHD). For example, EBV-associated diseases include, but are not limited to, Stevens-Johnson syndrome, post-transplant lymphoproliferative disorder, chronic fatigue syndrome, Burkitt's lymphoma and nasopharyngeal carcinoma. Initial CMV infection, which may have few symptoms, is always followed by a prolonged, inapparent infection during which the virus resides in cells without causing detectable damage or clinical illness. Severe impairment of the body's immune system by medication or disease may reactivate the virus from the latent or dormant state. CMV infection can also be life threatening for patients who are immunocompromised (e.g. patients with HIV, organ transplant recipients, or neonates). CMV-associated diseases include, for example, organ rejection, retinitis and restenosis. HHV-6-associated diseases include, for example, multiple sclerosis, organ rejection and lymphoproliferative and neoplastic disorders. HHV-7 associated diseases include, for example, organ rejection, lymphoproliferative and neoplastic disorders.

As used herein the terms treatment, treat or treating refers to a method of reducing the effects of a disease or condition or symptom of the disease or condition. Thus in the disclosed method treatment can refer to a 10%, 20%, 30%, 40%, 50%, 60%, 70%, 80%, 90% or 100% reduction in the severity of an established disease or condition or symptom of the disease or condition. For example, the method for treating a proliferative disorder is considered to be a treatment if there is a 10% reduction in one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, the terms prevent, preventing and prevention of a disease or disorder refers to an action, for example, administration of a therapeutic agent, that occurs before a subject begins to suffer from one or more symptoms of the disease or disorder, which inhibits or delays onset of the severity of one or more symptoms of the disease or disorder.

As used herein, subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, as well as fetuses, whether male or female, are intended to be covered. As used herein, patient or subject may be used interchangeably and can refer to a subject afflicted with a disease or disorder. The term patient or subject includes human and veterinary subjects.

### I. Viral Kinase Inhibitors

As used herein, a UL97 refers to UL97 kinase from cytomegalovirus (CMV) and homologs, variants and isoforms thereof. Homologs of UL97 kinase include HSV UL13, EBV BGLF4, HHV-6A UL69, HHV-6B UL69 and HHV-7 UL69. There are a variety of sequences that are disclosed on Genbank, at www.pubmed.gov, and these sequences and others are herein incorporated by reference in their entireties as well as for individual subsequences contained therein. For example, the amino acid and nucleic acid sequences of human CMV UL97 can be found at GenBank Accession Nos. NP_{—}040032.1 and NC_{—}001347.2, respectively. The amino acid and nucleic acid sequences of chimpanzee CMV can be found at GenBank Accession Nos. NP_{—}612729 and NC_{—}003521.1, respectively. The amino acid and nucleic acid sequences of HHV-6A U69 can be found at GenBank Accession Nos. NP_{—}042962.1 and NC_{—}001664.1, respectively. The amino acid and nucleic acid sequences of HHV-6B U69 can be found at GenBank Accession Nos. AAD49670 and AF157706.1, respectively. The amino acid and nucleic acid sequences of HHV-7 U69 can be found at GenBank Accession Nos. YP_{—}073809.1 and NC_{—}001716.2, respectively. The amino acid and nucleic acid sequences of EBV BGLF4 can be found at GenBank Accession Nos. ABB89262 and DQ279927.1, respectively.

Provided herein are viral kinase inhibitors for the treatment of proliferative disorders associated with herpesviruses. Specifically, provided are inhibitors of UL97 or a homolog of UL97 that prevent inactivation of the tumor suppressor protein Rb or a family member of the Rb family including p130 or p107. Inhibitors of a UL97 or a UL97 homolog include, but are not limited to, inhibitory peptides, drugs, functional nucleic acids and antibodies.

A. Inhibitory Peptides

Inhibitors of UL97 or UL97 homologs include inhibitory peptides or polypeptides. As used herein, the term peptide, polypeptide, protein or peptide portion is used broadly herein to mean two or more amino acids linked by a peptide bond. Protein, peptide and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide of the invention can contain up to several amino acid residues or more.

For example, the peptide that inhibits the UL97 or UL97 homolog kinase binds to the Rb binding domain of the UL97 or UL97 homolog. Inhibitory peptides include peptides that bind the LxCxE (SEQ ID NO:1) motif, the DSSE (SEQ ID NO:13) motif and/or the LxCxD (SEQ ID NO:2) motif of a UL97 or a UL97 homolog. Peptides can be tested for their ability to bind the Rb binding domain of a UL97 or a UL97 homolog by methods known to those of skill in the art, such as, for example, phage display and yeast two-hybrid assays.

Inhibitory peptides also include dominant negative mutants of a UL97 or a UL97 homolog. Dominant negative mutations (also called antimorphic mutations) have an altered phenotype that acts antagonistically to the wild-type or normal protein. Thus, dominant negative mutants of UL97 act to inhibit the normal UL97 protein. Such mutants can be generated, for example, by site directed mutagenesis or random mutagenesis. Proteins with a dominant negative phenotype can be screened for using methods known to those of skill in the art, for example, by phage display.

Nucleic acids that encode the aforementioned peptide sequences are also disclosed. These sequences include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. A wide variety of expression systems may be used to produce peptides as well as fragments, isoforms, and variants. Such peptides are selected based on their ability to bind the Rb binding domain of a UL97 or a UL97 homolog.

B. Drugs

Inhibitors of a UL97 or a UL97 homolog include, but are not limited to, quinazoline compounds, indolocarbazoles, benzimidazole L-riboside, maribavir (MBV), a derivative of ganciclovir, roscovitine, staurosporine, wortmannin, BIRB796, fasudil, flavopiridol, indurubin, NGIC-I and Go6976. Since UL97 is a cdc2 homolog, inhibitors of cdc2 can also be used in the provided methods. Inhibitors of UL97 are described in, for example, Herget et al., Antimicrobial Agents and Chemotherapy, 48(11):4154-4162 (2004), U.S. Pat. Nos. 7,105,529, 6,268,391 and 6,541,503.

C. Inhibitory Nucleic Acids

Also provided herein are functional nucleic acids that inhibit expression of UL97 or a UL97 homolog. Such functional nucleic acids include but are not limited to antisense molecules, aptamers, ribozymes, triplex forming molecules, RNA interference (RNAi), and external guide sequences. Thus, for example, a small interfering RNA (siRNA) could be used to reduce or eliminate expression of UL97 or a UL97 homolog. Examples of siRNA molecules that inhibit UL97 are described in Shin et al., Acta Virol. 50(4):263-8 (2006).

Functional nucleic acids are nucleic acid molecules that have a specific function, such as binding a target molecule or catalyzing a specific reaction. Functional nucleic acid molecules can interact with any macromolecule, such as DNA, RNA, polypeptides, or carbohydrate chains. Thus, functional nucleic acids can interact with the mRNA, genomic DNA, or polypeptide. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as hammerhead ribozymes, hairpin ribozymes and tetrahymena ribozymes). There are also a number of ribozymes that are not found in natural systems, but which have been engineered to catalyze specific reactions de novo (for example, but not limited to U.S. Pat. Nos. 5,807,718, and 5,910,408). Ribozymes may cleave RNA or DNA substrates. Representative examples of how to make and use ribozymes to catalyze a variety of different reactions can be found in U.S. Pat. Nos. 5,837,855, 5,877,022, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. Representative examples of how to make and use triplex forming molecules to bind a variety of different target molecules can be found in U.S. Pat. Nos. 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit (Ambion, Austin, Tex.).

D. Antibodies

Proteins that inhibit UL97 or UL97 homologs also include antibodies with antagonistic or inhibitory properties. Such antibodies are preferably antibodies that bind the receptor itself. Specifically, antibodies that bind the LxCxE (SEQ ID NO:1) motif, the DSSE (SEQ ID NO:13) motif and/or the LxCxD (SEQ ID NO:2) motif. In addition to intact immunoglobulin molecules, fragments, chimeras, or polymers of immunoglobulin molecules are also useful in the methods taught herein, as long as they are chosen for their ability to inhibit UL97 or UL97 homologs. The antibodies can be tested for their desired activity using in vitro assays, or by analogous methods, after which their in vivo therapeutic or prophylactic activities are tested according to known clinical testing methods.

The term antibody is used herein in a broad sense and includes both polyclonal and monoclonal antibodies. Monoclonal antibodies can be made using any procedure that produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

Digestion of antibodies to produce fragments thereof, e.g., Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibody fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curt Opin. Biotechnol. 3:348-354, 1992).

As used herein, the term antibody or antibodies can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1): δ 95, 1991). Human antibodies (and fragments thereof) can also be produced using phage display libraries (Hoogenboom et al., J. Mol. Biol., 227:381, 1991; Marks et al., J. Mol. Biol., 222:581, 1991). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551 255 (1993); Jakobovits et al., Nature, 362:255 258 (1993); Bruggermann et al., Year in Immunol., 7:33 (1993)). Specifically, the homozygous deletion of the antibody heavy chain joining region (J(H)) gene in these chimeric and germ line mutant mice results in complete inhibition of endogenous antibody production, and the successful transfer of the human germ line antibody gene array into such germ line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. As used throughout, antibody fragments include Fv, Fab, Fab′, or other antigen binding portion of an antibody. Methods for humanizing non human antibodies are well known in the art. For example, humanized antibodies can be generated according to the methods of Winter and co workers (Jones et al., Nature, 321:522 525 (1986), Riechmann et al., Nature, 332:323 327 (1988), Verhoeyen et al., Science, 239:1534 1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### II. Methods of Screening

Methods of screening for agents that inhibit the activity of a UL97 or a UL97 homolog are provided. Such a screening method comprises the steps of providing a cell that expresses a UL97 or a UL97 homolog, contacting the cell with a candidate agent to be tested and determining whether the candidate agent prevents the activation of Rb by the UL97 or the UL97 homolog. Another method of screening for agents that inhibit the activity of a UL97 or a UL97 homolog comprises the steps of providing a sample comprising a UL97 or a UL97 homolog and Rb, contacting the sample with a candidate agent to be tested and determining whether the candidate agent prevents the activation of Rb by the UL97 or the UL97 homolog.

The provided cells that express a UL97 or a UL97 homolog can be made by infecting the cell with a virus comprising a UL97 or a UL97 homolog wherein the UL97 or the UL97 homolog is expressed in the cell following infection. The cell can also be a prokaryotic or an eukaryotic cell that has been transfected with a nucleotide sequence encoding a UL97 or a UL97 homolog or a variant or a fragment thereof, operably linked to a promoter. Using DNA recombination techniques well known by the one skill in the art, protein encoding DNA sequences can be inserted into an expression vector, downstream from a promoter sequence.

Such methods allow one skilled in the art to select candidate agents that exert a regulating effect on the ability of a UL97 or a UL97 homolog to prevent activation of Rb. Such agents may be useful as active ingredients included in pharmaceutical compositions for treating patients suffering from herpesvirus associated disorders.

Methods for determining whether the candidate agent prevents activation of Rb by a UL97 or a UL97 homolog are well known to those of skill in the art. The assay can be, for example, a surrogate assay, a phosphorylation assay, a kinase assay, a high throughput phosphorylation assay, affinity-tagged phosphorylation assay by matrix-assisted laser desorption/ionization time-of-flight mass spectrometry or one of the provided methods described in the examples below.

### III. Compositions

Pharmaceutical compositions comprising one or more of the inhibitors or agents provided herein may include carriers, thickeners, diluents, buffers, preservatives, surface active agents and the like in addition to the molecule of choice. Pharmaceutical compositions may also include one or more active ingredients such as antimicrobial agent, a chemotherapeutic agent, and the like. The compositions of the present application can be administered in vivo in a pharmaceutically acceptable carrier. By pharmaceutically acceptable is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The disclosed compositions can be administered in a number of ways depending on whether local or systemic treatment is desired, and on the area to be treated. Thus, the disclosed compositions can be administered, for example, orally, parenterally (e.g., intravenously), by intramuscular injection, by intraperitoneal injection, transdermally, extracorporeally, or topically. For example, if the subject has a neoplastic disorder or cancer, the compositions may be administered locally at or near the site of the tumor.

The materials may be in solution, suspension (for example, incorporated into microparticles, liposomes, or cells). These may be targeted to a particular cell type via antibodies, receptors, or receptor ligands. Suitable carriers and their formulations are described in Remington: The Science and Practice of Pharmacy (21th ed.) ed. David B. Troy, Lippincott Williams & Wilkins, 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of composition being administered.

The terms effective amount and effective dosage are used interchangeably. The term effective amount is defined as any amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like. Generally, the dosage will vary with the age, condition, sex, type of disease and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. The provided compositions can be administered in combination with one or more other therapeutic or prophylactic regimens. As used throughout, a therapeutic agent is a compound or composition effective in ameliorating a pathological condition. Illustrative examples of therapeutic agents include, but are not limited to, an anti-cancer compound, anti-inflammatory agents, anti-viral agents, anti-retroviral agents, anti-opportunistic agents, antibiotics, immunosuppressive agents, immunoglobulins, and antimalarial agents.

An anti-cancer compound or chemotherapeutic agent is a compound or composition effective in inhibiting or arresting the growth of an abnormally growing cell. Thus, such an agent may be used therapeutically to treat cancer as well as other diseases marked by abnormal cell growth. A pharmaceutically effective amount of an anti-cancer compound is an amount administered to an individual sufficient to cause inhibition or arrest of the growth of an abnormally growing cell. Illustrative examples of anti-cancer compounds include: bleomycin, carboplatin, chlorambucil, cisplatin, colchicine, cyclophosphamide, daunorubicin, dactinomycin, diethylstilbestrol doxorubicin, etoposide, 5-fluorouracil, floxuridine, melphalan, methotrexate, mitomycin, 6-mercaptopurine, teniposide, 6-thioguanine, vincristine and vinblastine.

The provided compositions may be administered in combination with an antiviral agent. Antiviral agents that may be administered include, but are not limited to, acyclovir, ribavirin, amantadine, remantidine, ganciclovir, foscarnet, and cidofovir.

Antiretroviral agents, nucleoside reverse transcriptase inhibitors, non-nucleoside reverse transcriptase inhibitors, and/or protease inhibitors may be used in any combination with the provided compositions to treat AIDS and/or prevent or treat HIV infection.

The provided compositions may be administered in combination with anti-opportunistic infection agents. Anti-opportunistic agents include, but are not limited to, trimethoprim-pentamidine, sulfamethoxazole, DAPSONES (Jacobus Pharmaceuticals, Princeton, N.J.), ATOVAQUONE® (GlaxoSMithKline, Research Triangle Park, N.C.), ISONIAZID® (CIBA Pharmaceuticals, Summit, N.J.), RIFADIN® (rifampin)(Hoechst-Marrion-Roussel, Kansas City, Mo.), PYRAZINAMIDE® (Ledelrle, Pearl River, N.Y.), BIAXIN® (clarithromycin) (Abbott Laboratories, Chicago, Ill.), ETHAMBUTOL® (Ledelrle, Pearl River, N.Y.), RIFABUTIN® (Pharmacia & Upjohn Company, Kalamazoo, Mich.), AZITHROMYCIN® (Pfizer Inc., NewYork, N.Y.), GANCICLOVIR (Roche Pharmaceuticals, Nutley, N.J.), FOSCARNET® (Astra, Westborough, Mass.), CIDOFOVIR® (Gilead Sciences, Foster City, Calif.), KETOCONAZOLE® (Janssen, Titusville, N.J.), FLUCONAZOLE® (Pfizer Inc., NewYork, N.Y.), ITRACONAZOLE® (Janssen, Titusville, N.J.), ACYCLOVIRS (Glaxo-Wellcome, Research Triangle Park, N.C.), FAMCICOLCIR® (SmithKline Beecham Pharmaceuticals, Pittsburgh, Pa.), pyrimethamine, leucovorin, NEUPOGEN® (filgrastim/GM-CSF) (Amgen, Thousand Oaks, Calif.), and LEUKINE® (sargramostim/GM-CSF) (Immunex, Seattle, Wash.).

Antibacterial agents that may be administered with the provided compositions include, but are not limited to, amoxicillin, aminoglycosides, beta-lactam (glycopeptide), betalactamases, clindamycin, chloramphenicol, cephalosporins, ciprofloxacin, erythromycin, fluoroquinolones, macrolides, metronidazole, penicillins, quinolones, ritampin, streptomycin, sulfonamide, tetracyclines, trimethoprim, trimethoprim-sulfamthoxazole, and vancomycin.

Immunosuppressive agents, that may be administered in combination with the provided compositions include, but are not limited to steroids, cyclosporine, cyclosporine analogs, cyclophosphamide methylprednisone, prednisone, azathioprine, FK-506 (Fujisawa Pharmaceuticals, Deerfield, Ill.), 15 deoxyspergualin, and other immunosuppressive agents that act by suppressing the function of responding immune cells (including, for example, T cells), directly (e.g., by acting on the immune cell) or indirectly (by acting on other mediating cells). Immunosuppresive agents also include, ORTHOCLONE® (OKT3) (Ortho Biotech, Raritan, N.J.), SANDIMMUNE®ORAL (cyclosporine) (Sandoz Pharmaceuticals, Hanover, N.J.), PROGRAF® (tacrolimus) (Fujisawa Pharmaceuticals, Deerfield, Ill.), CELLCEPT® (mycophenolate) (Roche Pharmaceuticals, Nutley, N.J.), azathioprine, glucorticosteroids, and RAPAMUNE® (sirolimus) (Wyeth, Collegeville, Pa.). The immunosuppressants in combination with the provided inhibitors may be used to prevent rejection of organ transplantation.

Anti-inflammatory agents that may be administered with the provided compositions include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, ninesulide, orgotein, oxaceprol, paranyline, perisoxal, pifoxime, proquazone, proxazole, and tenidap.

Any of the aforementioned treatments can be used in any combination with the compositions described herein. Thus, for example, the compositions can be administered in combination with a chemotherapeutic agent and radiation. Other combinations can be administered as desired by those of skill in the art. Combinations may be administered either concomitantly (e.g., as an admixture), separately but simultaneously (e.g., via separate intravenous lines into the same subject), or sequentially (e.g., one of the compounds or agents is given first followed by the second). Thus, the term combination is used to refer to either concomitant, simultaneous, or sequential administration of two or more agents.

### IV. Medical Devices

Also provided are medical devices comprising an implantable medical device and an inhibitor of a UL97 or a UL97 homolog. Implantable medical devices, such as drug delivery systems, pacemakers, artificial joints, and organs play an important role in health care. As used herein, medical device refers broadly to any exogenous material or device which is inserted within the body of a vertebrate, e.g., bird, reptile, amphibian, or mammal.

Medical devices include, tissue engineering scaffold, guided tissue repair material, wound dressing, drug delivery vehicle, anti-adhesion material, cell encapsulation material, coating, implant, stent, orthopaedic device, prosthetic, adhesives, diagnostics, sutures, surgical meshes, staples, meniscus repair and regeneration devices, screws (interference screws and meniscal screws), bone plates and plating systems, cardiovascular patches, pericardial patches, slings, pins, anti-adhesion barriers, articular cartilage repair devices, nerve guides, tendon and ligament repair devices, atrial septal defect pathces, bulking and filling agents, vein valves, bone marrow scaffolds, bone graft scaffolds, skin substitutes, dural substitutes, ocular implants, spinal fusion cages, and muscular implants (cardiac and skelatal). Preferably, the medical device is an implant, a scaffold, a prosthesis, a heart valve, vascular graft, pacemaker, stent, catheter, an intravenous tube or a drug delivery device. The drug delivery device can provide for sustained release of the inhibitor. Implantable drug delivery systems are disclosed in U.S. Pat. Nos. 3,773,919, 4,155,992, 4,379,138, 4,130,639, 4,900,556, 4,186,189, 5,593,697, and 5,342,622. These devices may include additives or other materials. Additives and other materials may include those components added for the purpose of further modification of a particular property or properties, and/or those components which are biologically active such as cell attachment factors, growth factors, peptides, antibodies and their fragments. The device can be administered to a subject with, for example, an organ transplant, neoplastic disease, restenosis, heart disease.

Inhibitors of a UL97 or a UL97 homolog include, but are not limited to, inhibitory peptides, drugs, functional nucleic acids and antibodies. Preferably, the inhibitor of a UL97 or a UL97 homolog is an inhibitory peptide selected from the group consisting of an inhibitory peptide that binds the LxCxE (SEQ ID NO:1) motif of a UL97 or a UL97 homolog, an inhibitory peptide that binds the LxCxD (SEQ ID NO:2) motif of a UL97 or a UL97 homolog, an inhibitory peptide that binds the LxCxE (SEQ ID NO:1) and LxCxD (SEQ ID NO:2) motifs of a UL97 or a UL97 homolog and an inhibitory peptide that binds the LxCxE (SEQ ID NO:1) and the DSSE (SEQ ID NO:13) motifs of a UL97 or a UL97 homolog.

Determination of the precise tissue/implant configuration and the quantity and form of tissue response modifier effective to control the tissue response at the site of implantation is within the abilities of one of ordinary skill in the art, and will depend on the particular site of implantation, the length of time that the implant is intended to remain in the body, and the implant itself.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a biomarker is disclosed and discussed and a number of modifications that can be made to a number of molecules including the biomarker are discussed, each and every combination and permutation of the biomarker and the modifications that are possible are specifically contemplated unless specifically indicated to the contrary. Thus, if a class of molecules A, B, and C are disclosed as well as a class of molecules D, E, and F and an example of a combination molecule, A-D, is disclosed, then even if each is not individually recited, each is individually and collectively contemplated. Thus, is this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Ranges may be expressed herein as from about one particular value, and/or to about another particular value. Similarly, when values are expressed as approximations, by use of the term about, it will be understood that the particular value is included. It will be further understood that the endpoints of each of the ranges are significant both in relation to the other endpoint, and independently of the other endpoint.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination). Publications cited herein and the material for which they are cited are hereby specifically incorporated by reference in their entireties.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting. Note the headings used herein are for organizational purposes only and are not meant to limit the description provided herein or the claims attached hereto.

### EXAMPLES

### Example 1

### UL97 Kinase Activity Required for Hyperphosphorylation of Rb and Inhibition of Nuclear Aggresomes

### Materials and Methods

Cells and Viruses. Human foreskin fibroblast (HFF) cells were established and routinely propagated in monolayer cultures in minimum Eagle's medium with Earle's salts supplemented with 10% fetal bovine serum (FBS), 2 mM L-glutamine, 100 μg/ml penicillin G and 25 μg/ml gentamicin. HCMV strain AD169 was obtained from the American Type Culture Collection (Manassas, Va.) and virus stocks were prepared and titered as described previously (Prichard et al., Antimicrob. Agents Chemother. 50:1336-41 (2006)). The construction and characterization of a UL97 null mutant (RCΔ97.08) was described previously (Prichard et al., J. Virol. 73:5663-70 (1999). Maribavir was obtained through the Antiviral Substances Program of the National Institute for Allergy and Infectious Diseases.

Plasmids. Construction of the plasmids expressing the pp65-GFP fusion protein as well as those for the epitope tagged versions of the wt UL97 ORF and the K355M mutation were described previously (Prichard et al., J. Virol. 79:15494-502 (2005)). Mutation of the LxCxD (SEQ ID NO:2) motif was performed by amplifying the UL97 ORF of AD169 using UL97 forward primer 5′-CAC CAT GTC CTC CGC ACT TCG GTC T-3′ (SEQ ID NO:14) and LxCxD (SEQ ID NO:2) reverse primer 5′-TCA CCT TCG ACC GCC CGT AGC TGT CGA TG-3′ (SEQ ID NO:15), and UL97 reverse primer 5′-TTA CTC GGG GAA CAG TTG G-3′ (SEQ ID NO:16) with LxCxD (SEQ ID NO:2) forward primer 5′-AGT GGA AGC TGG CGG GCA TCG ACA GCT AC-3′ (SEQ ID NO:17). The two PCR products were gel purified, mixed in an equimolar ratio and used as a template for PCR using the UL97 forward primer and UL97 reverse primer. The PCR product was cloned into the pENTR/D/TOPO vector (Invitrogen, Carlsbad, Calif.) to yield pMP263. The C428G mutation was confirmed by sequencing and recombined into pcDNA3 V5-DEST (Invitrogen, Carlsbad, Calif.) to yield pMP 264. Mutation of the LxCxE (SEQ ID NO:1) motif was performed in a similar manner using the UL97 forward primer with LxCxE reverse primer 5′-GGT GCC GAA CGC GCC GGC GCT TTG AAG-3′ (SEQ ID NO:18), and UL97 reverse primer with LxCxE forward primer 5′-CCA CGG CTT GCG CGG CCG CGAAAC TTC-3′ (SEQ ID NO:19). The two PCR products were combined and used as a template for a second PCR using the UL97 forward primer and UL97 reverse primer. The product was cloned into pENTR/D/TOPO (pMP 265) and the C151G mutation was confirmed by sequence analysis. The open reading frame containing the mutation was them recombined into the pcDNA3 V5-DEST vector to yield pMP 269.

Polyacrylamide gels and western blotting. HFF cells were infected at a multiplicity of infection of 2 PFU/cell and at 24 and 72 hours following infection they were disrupted in 2× Lamelli buffer (Sigma-Aldrich, St. Louis, Mo.) and separated on 10%, 7.5%, or 5% polyacrylamide gels depending on the size of the protein to be resolved (BioRad, Hercules, Calif.). Separated proteins were transferred to PVDF membranes (Roche Applied Science, Indianapolis, Ind.) in a buffer containing 28 mM Tris, 39 mM glycine, 0.0375% SDS and 20% methanol in a semi dry transfer cell (BioRad, Hercules, Calif.). Membranes were blocked in 1% blocking buffer (Roche Applied Science, Indianapolis, Ind.), incubated with primary antibodies overnight at 4° C. and washed extensively with PBS supplemented with 0.02% tween 20. A secondary antibody conjugated to alkaline phosphatase (Southern Biotechnology Associates, Birmingham, Ala.) and CDP* (Roche Applied Science, Indianapolis, Ind.) were used to detect the bound primary antibody. Monoclonal antibodies used in these studies were directed against PML (H-238) (Santa Cruz Biotechnology, Santa Cruz, Calif.), DAXX (Upstate Cell Signaling Solutions, Tamecula, Calif.), p53, retinoblastoma, β-tubulin, CREB binding protein (CBP) (Chemicon International, Tamecula, Calif.), and both V5 and Xpress (Invitrogen, Carlsbad, Calif.).

Indirect immunofluorescence microscopy. Cells expressing proteins from transfected plasmids were visualized by methods published previously (Prichard et al., J. Virol. 79:15494-502 (2005)). Briefly, monolayers of HFF cells were grown on 12 mm diameter coverslips in 24-well plates. Transfected cells were fixed for 15 minutes with freshly prepared 2% formaldehyde in phosphate buffered saline (PBS), washed two times with PBS, and membranes were permeabilized with 0.2% Triton X-100 in PBS for 15 minutes. Monoclonal antibody to the V5 epitope tag was purchased from Invitrogen (Carlsbad, Calif.). Monoclonal antibodies to IE1 (63-27) and ppUL44 (28-21) were generously provided by Bill Britt and were used as culture supernatants with goat anti-mouse secondary antibodies conjugated to FITC or Texas Red (Southern Biotechnology, Birmingham, Ala.).

Isolation of nuclear and cytoplasmic aggresomes. Low passage HFF cells were infected with RCΔ97.08 at a low MOI in 175 cm² flasks, and infected cells were passaged at 7 days post infection as plaques started to form as well as 12 and 16 dpi until 100% CPE was observed. Infected cells were dislodged with 0.25% trypisn-EDTA (Gibco, Grand Island, N.Y.) and resuspended in a volume of 10 ml growth medium. Cells were collected by centrifugation and resuspended in PBS with the addition of 0.6% NP-40 and nuclei were centrifuged through a cushion of histopaque 1077 (Sigma Chemical Company, St. Louis, Mo.) at 1000×g for 5 minutes. Nuclei were lysed in PBS supplemented with 2.5 M NaCl and cellular DNA was degraded with 10,000 units of deoxyribonuclease I. An equal volume of 5 M urea was added to the nuclear lysate and the nuclear aggresomes were collected by centrifugation at 3500×g for 10 minutes through a histopaque cushion. Nuclear aggresomes were resuspended in PBS supplemented with 0.5% NP-40 and frozen at −80° C. Cytoplasmic aggresomes were isolated from the cytoplasmic fraction by sedimentation at 3500×g. The sedimented material was resuspended in a PBS buffer containing 5M urea and aggresomes were collected by sedimentation through a histopaque cushion and resuspended in PBS with 0.5% NP-40 and frozen at −80° C.

Immunoprecipitation. COST cells in 75 cm² flasks were transfected with plasmids expressing epitope tagged versions of pUL97 and two negative controls, UL89 and UL104. Two days post transfection cells were collected by centrifugation and lysed in 1 ml RIPA buffer (150 mM NaCl, 1% NP-40, 0.5% deoxycholate, 0.1% SDS, 50 mM Tris pH 7.5). Cell lysates were clarified at 10,000×g and 50 μL of Protein A sepharose bound to the V5 monoclonal antibody was added to each supernatant. The suspension was incubated for 12 h at 4° C., the beads were washed twice with RIPA buffer and frozen until further use. Bound proteins on 10 μl of beads were added to a tube containing 100 μl of PBS and 25 μg of a recombinant protein consisting of the carboxyl terminus of Rb fused to the maltose binding protein was added to each tube (Cell Signaling Technology, Beverly Mass.). The suspension was incubated for 1 h at 37° C. and the beads were washed three times in PBS. Proteins bound to the beads were eluted with Lamelli buffer, separated on 7.5% SDS PAGE gels and transferred to a PVDF membrane. Proteins were detected with the monoclonal antibody to the V5 epitope and also with a rabbit polyclonal antisera directed against Rb (Neomarkers, Freemont, Calif.).

Tryptic Digestion of Viral Inclusions. HCMV inclusion bodies were denatured by the addition of urea to 8M and heating to 37° C. for 30 min. The sample was then diluted 4-fold with 100 mM ammonium bicarbonate (AB) and CaCl2 was added to 1 mM. Methylated, sequencing-grade porcine trypsin (Promega, Madison, Wis.) was added at a substrate-to-enzyme ratio of 50:1 (mass:mass) and incubated at 37° C. for 15 hours. Sample cleanup was achieved using a 1-mL SPE C18 column (Supelco, Bellefonte, Pa.). The peptides were eluted from each column with 1 mL of methanol and concentrated via SpeedVac. The samples were reconstituted to 10 μg/μL with 25 mM ammonium bicarbonate and frozen at −20° C. until analyzed.

Tandem Mass Spectrometric Analysis of Peptides. Peptide samples were analyzed by reversed phase cLC coupled directly with electrospray tandem mass spectrometers (Thermo Finnigan, models LCQ™ Duo and LCQ™ DecaXP, San Jose, Calif.). Chromatography was performed on a 60-cm, 150 μm i.d.×360 μm o.d capillary column (Polymicro Technologies, Phoenix, Ariz.) packed with Jupiter C18 5-μm-diameter particles (Phenomenex, Torrence, Calif.). A solvent gradient was used to elute the peptides using 0.1% formic acid in water (A) and 0.1% formic acid in acetonitrile (B). The gradient was linear from 0 to 5% solvent B in 20 minutes, followed by 5 to 70% solvent B in 80 minutes, and then 70-85% solvent B in 45 minutes. Solvent flow rate was 1.8 μl/min. The capillary LC system was coupled to a LCQ ion trap mass spectrometer (Thermo Finnigan, San Jose, Calif.) using an in-house manufactured ESI interface in which no sheath gas or makeup liquid was used. The temperature of heated capillary and electrospray voltage was 200° C. and 3.0 kV, respectively. Samples were analyzed using the data-dependent MS/MS mode over the m/z range of 300-2000. The three most abundant ions detected in each MS scan were selected for collision-induced dissociation.

SEQUEST Analysis. The SEQUEST algorithm was run on each of the datasets against a combined database comprised of the HCMV.fasta and the human.fasta from the National Center for Biotechnology Information. Tandem MS peaks were generated by extract_msn.exe, part of the SEQUEST software package. A peptide was considered to be a match by using a conservative criteria set developed by Yates and coworkers (Link et al, 1999; Washburn et al., 2001). Briefly, all accepted SEQUEST results had a delta Cn of 0.1 or greater. Peptides with a +1 charge state were accepted if they were fully tryptic and had a cross correlation (XCorr) of at least 1.9. Peptides with a +2 charge state were accepted if they were fully tryptic or partially tryptic and had an XCorr of at least 2.2. Peptides with +2 or +3 charge states with an XCorr of at least 3.0 or 3.75, respectively, were accepted regardless of their tryptic state.

### Results

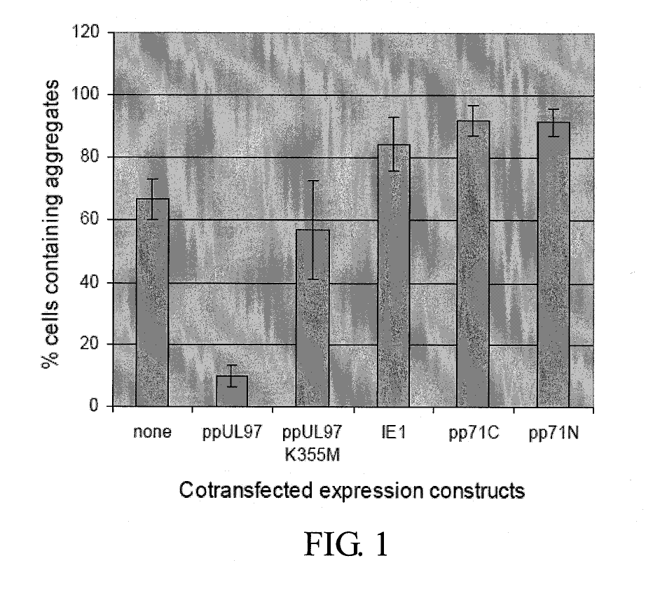

Aggregation of pp65 is affected by pp71 and IE1. Inappropriate aggregation of viral proteins occurs in the absence of the UL97 kinase. It was reported previously, that expression of a pp65-GFP fusion protein in COST cells was sufficient to induce formation of large nuclear aggregates, which were inhibited by the coexpression of pUL97 (Prichard et al., J. Virol. 79:15494-502 (2005)). Expression plasmids for 128 viral open reading frames were individually cotransfected with a plasmid expressing GFP-tagged pp65 and the effect on aggregation was assessed by immunofluoresence microscopy. Plasmids expressing either pp71 or IE1 significantly increased the formation of pp65 aggregates and did not appear to be related to expression levels since cotransfection with a plasmid expressing both IE1 and IE2 had no effect. Confirmatory transfections demonstrated that expression of both IE1 and pp71 significantly increased the proportion of pp65 expressing cells that exhibited more than two large nuclear aggregates (FIG. 1). Both proteins have been reported to interact with components of PML domains and also interact functionally with Rb. PML bodies have also been reported to be involved in protein aggregation through the formation of nuclear aggresomes, which are cellular structures thought to be involved in the sequestration of misfolded proteins. These data taken together indicate that UL97 affects aggregate formation by altering cellular functions associated with PML bodies.

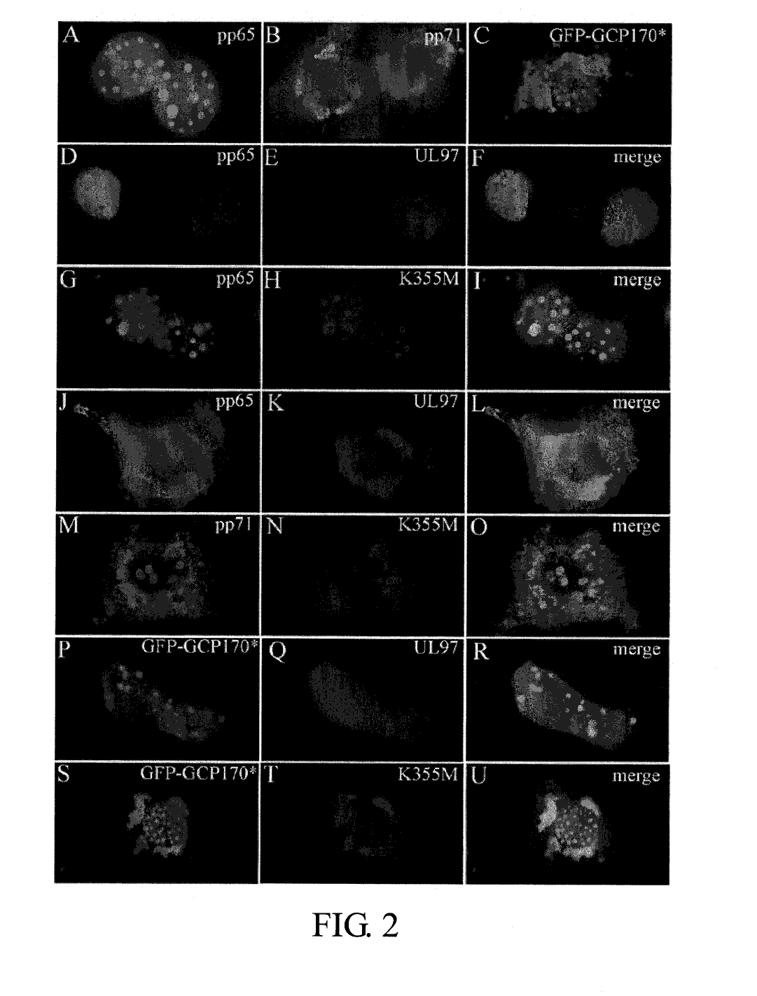

Sequestration of Proteins in Nuclear Aggresomes is Inhibited by the Kinase. Reports linking PML bodies and the nuclear aggregation prompted an examination of the effect of the UL97 kinase on the aggregation of two other proteins in the nucleus. The pp71 tegument protein was reported to form large nuclear aggregates similar to those formed by pp65 and was affected by the presence of PML. A cellular protein, GFP-GCP170*, also induces large aggregates called nuclear aggresomes and been used as a marker for these structures. Transfection of COS7 cells with epitope tagged versions of pp71 confirmed that it formed large nuclear aggregates (FIG. 2B) that resembled those formed by pp65 (FIG. 2A), and cytoplasmic aggregations were also observed that contained pp71. The expression of GFP-GCP 170* was also sufficient to induce cytoplasmic and nuclear aggresomes in COS7 (FIG. 2C). The nuclear aggregates with the viral proteins and pp65 were morphologically similar to the nuclear aggresomes induced by GFP-GCP170* and the cytoplasmic aggregates induced with pp71 also resembled the cytoplasmic aggresomes. To characterize the effect of the kinase on the aggregation of these proteins, the kinase and a K355M mutant without enzymatic activity were coexpressed with pp65, pp71 and GFP-GCP170*. The kinase inhibited the formation of pp65 aggregates (FIG. 2 D-F), whereas the kinase negative form of UL97 (K355M) was unable to inhibit their formation and was recruited to the large nuclear aggregates (FIG. 2 G-I). The inhibition of aggregation formation by the kinase was also antagonized by Maribavir (MBV), a specific inhibitor of its kinase activity. The kinase also inhibited the aggregation of pp71 in the nucleus of cotransfected cells and both proteins remained in the cytoplasm (FIG. 2 J-L). The K355M mutant was unable to inhibit the aggregation of pp71 and was recruited to both nuclear and cytoplasmic aggregates (FIG. 2 M-O). The kinase was also unable to inhibit the aggregation of pp71 in the presence of MBV, confirming that the inhibition was dependent on its enzymatic activity.

These results indicate that the ability of the kinase to inhibit nuclear aggregates was not limited to pp65 but also inhibited the aggregation of another viral phosphoproteins. The kinase effect on general cellular function and on nuclear aggresomes induced by GFP-GCP170* was examined. Cotransfection with plasmids expressing the kinase reduced the size and number of both nuclear and cytoplasmic aggresomes and also affected their distribution in the cell (FIG. 2 P-R). This activity appeared to be dependent on its kinase activity since the K355M mutant failed to inhibit their formation (FIG. 2 S-T) and rather, was recruited to cytoplasmic aggresomes. Similar results were also observed when the active kinase was inhibited with MBV. These data indicate that the kinase inhibits nuclear aggregation and the formation of both cytoplasmic and nuclear aggresomes. They indicate also, that the aggregation of the virion tegument proteins in the nucleus relates to the formation of nuclear aggresomes.

Tegument aggregates are nuclear aggresomes containing virion proteins. One interpretation of the data was that virion tegument proteins expressed in uninfected cells were inside nuclear aggresomes and that their disruption by the kinase prevented the cell from sequestering these proteins. To test this hypothesis, GFP-GCP170* was co-expressed with virion tegument proteins to determine if they colocalized. Each of the tegument proteins, pp65, pp71, and pUL69 localized with GFP-GCP 170*, indicating that the aggregation observed with these proteins was mediated by the aggresomes. Therefore, the structures referred to as tegument aggregates are nuclear aggresomes that contain proteins. The data also indicate that inhibition of tegument protein aggregation is related to the ability of the kinase to prevent the formation of these cellular structures.

Aggresomes contain large quantities of virion proteins. To determine the protein content of the HCMV nuclear and cytoplasmic aggresomes, they were purified from infected cells by methods described previously and subjected to an analysis by mass spectrometry (Prichard et al., J. Virol. 79:15494-502 (2005); Varnum et al., J. Virol. 78:10960-6 (2004)). The aggresomes were denatured and digested with trypsin. To identify the viral proteins found in the aggresomes, the complex mixture of peptides was analyzed by two-dimensional liquid chromatography coupled to MS/MS and the results were compared to a HCMV-FASTA database. This analysis revealed that the aggresomes contained large quantities of viral structural proteins (Table 1).

TABLE 1
Viral Proteins Identified by LC/MS/MS in Cytoplasmic and Nuclear
Aggresomes.

| | Cytoplasmic | | Nuclear | | |
|---|---|---|---|---|---|
| | Aggresomes | | Aggresomes | | |
| | | No. of | | No. of | |
| HCMV | Max | unique | Max | unique | |
| ORF | X_{corr} | Peptides | X_{corr} | Peptides | Description |
| IRS1 | 5.12 | 2 | 4.73 | 3 | Transcriptional transactivator; |
| | | | | | US22 family member |
| UL25 | 4.97 | 15 | 5.54 | 25 | Tegument protein; UL25 |
| | | | | | family member |
| UL26 | 3.46 | 2 | 4.91 | 4 | Tegument protein; US22 |
| | | | | | family member |
| UL31 | N.D. | N > D. | 3.97 | 3 | Hypothetical protein |
| UL32 | 4.71 | 4 | 4.38 | 4 | Pp150 tegument protein |
| UL34 | N.D. | N.D. | 4.34 | 2 | Transcriptional repressor |
| UL35 | N.D. | N.D. | 6.58 | 3 | UL25 family member |
| UL44 | 5.57 | 11 | 5.16 | 7 | Processivity subunit of DNA |
| | | | | | polymerase; HSV-1 UL42 |
| | | | | | counterpart |
| UL46 | N.D. | N.D. | 4.18 | 3 | Intercapsomeric triplex capsid |
| | | | | | protein; HSV-1 UL38 |
| | | | | | counterpart |
| UL47 | N.D. | N.D. | 3.50 | 1 | Tegument protein; |
| | | | | | HSV-1UL37 counterpart |
| UL48 | 4.30 | 1 | 5.68 | 5 | Tegument protein; |
| | | | | | HSV-1 UL36 counterpart |
| UL48A | 7.17 | 5 | 7.03 | 2 | Capsid protein located at tips |
| | | | | | of hexons; HSV-1 UL35 |
| | | | | | counterpart |
| UL50 | 6.23 | 1 | N.D. | N.D. | Membrane-associated protein |
| | | | | | involved in nuclear capsid |
| | | | | | egress; HSV-1 UL34 |
| | | | | | counterpart |
| UL57 | 1.91 | 1 | N.D. | N.D. | Single-stranded DNA-binding |
| | | | | | protein; HSV-1 UL29 |
| | | | | | counterpart |
| UL69 | N.D. | N.D. | 3.28 | 2 | Post-transcriptional regulator |
| | | | | | of gene expression; |
| | | | | | HSV-1 UL54 counterpart |
| UL71 | 5.09 | 2 | N.D. | N.D. | Tegument protein; |
| | | | | | HSV-1 Ul51 counterpart |
| UL77 | N.D. | N.D. | 4.59 | 1 | DNA packaging protein; |
| | | | | | HSV-1 UL25 counterpart |
| UL80 | 5.60 | 9 | 4.10 | 2 | Protease (N-terminus) and |
| | | | | | minor scaffold protein |
| | | | | | (C-terminus); HSV0-1 UL26 |
| | | | | | counterpart |
| UL82 | N.D. | N.D. | 4.82 | 5 | Pp71 upper matrix |
| | | | | | phosphosprotein; tegument |
| | | | | | protein; transactivator |
| | | | | | of MIEP |
| UL83 | 6.00 | 60 | 6.56 | 82 | Pp65 lower matrix |
| | | | | | phosphosprotein; tegument |
| | | | | | protein |
| UL84 | 5.05 | 4 | 5.65 | 3 | Transdominant inhibitor of |
| | | | | | IE2-mediated |
| | | | | | transactivation |
| UL85 | 3.00 | 2 | 2.99 | 3 | Intercapsomeric triplex |
| | | | | | capsid protein; |
| | | | | | HSV-1 UL18 coutnerpart |
| UL94 | N.D. | N.D. | 3.83 | 1 | Tegument protein; |
| | | | | | HSV-1 UL16 counterpart |
| UL98 | N.D. | N.D. | 3.32 | 1 | DNase; HSV-1 UL12 |
| | | | | | coutnerpart |
| UL104 | N.D. | N.D. | 3.84 | 2 | DNA packaging protein; |
| | | | | | capsid portal protein; |
| | | | | | HSV-1 UL16 counterpart |
| UL112 | 1.97 | 1 | N.D. | N.D. | Hypothetical protein |
| UL115 | 4.16 | 1 | N.D. | N.D. | Envelope glycoprotein; |
| | | | | | associated with gH and gO; |
| | | | | | HSV-1 UL1 |
| | | | | | counterpart; gL |
| UL122 | 4.24 | 3 | 3.23 | 1 | Immediate-early |
| | | | | | transcriptional regulator; |
| | | | | | IE2 |
| US22 | 2.48 | 1 | 3.53 | 1 | Tegument protein; US22 |
| | | | | | family member |

N.D., not detected

There were 25 HCMV proteins in the nuclear aggresomes and 19 in the cytoplasmic aggresomes. These include the capsid proteins UL46 (minor capsid binding protein), UL48A (smallest capsid protein), UL80 (assembly protein), UL85 (minor capsid protein), and UL86 (major capsid protein), as well as a number of tegument proteins including UL25, UL26, UL32, UL35, UL47, UL48, UL82, UL83, UL94, and US22. In addition, a number of proteins involved in transcription and DNA replication were also present including IRS1, UL31, UL34, UL44, UL57, UL69, UL84, UL98, UL104, and UL122. Overall the ratios of viral proteins present in the aggresomes resemble dense bodies, rather than virions, showing that they are incorporated prior to genome packaging.

To identify the cellular proteins present in the aggresomes, the result from the mass spectroscopy analysis were compared to the predicted peptides of a human-FASTA database. The nuclear and cytoplasmic aggresomes contained a number of cellular heat shock proteins including HSP70, HSP71c, HSP70-2, and HSP-60 (Table 2).

TABLE 2
Notable Cellular Proteins Identified by LC/MS/MS in Cytoplasmic and
Nuclear Aggresomes.

| | | | Max |
|---|---|---|---|
| | Reference | Description | X_{corr} |
| Cytoplasmic | gil5729877 | Heat shock 70 kD protein 10 | 4.74 |
| Aggresomes | | (HSC71) | |
| | gil4758570 | Heat shock 70 kD protein 9B | 5.11 |
| | gil1708307 | Heat shock-related 70 kD protein 2 | 3.94 |
| | gil129379 | Mitochondrial matrix protein p1 | 3.93 |
| | | precursor (HSP-60) | |
| | gil125731 | ATP-dependent DNA Helicase II, | 7.14 |
| | | 80 kDa subunit | |
| | gil114762 | Nucleophosmin (nucleolar | 6.12 |
| | | phosphoprotein B23) | |
| Nuclear | gil7446411 | Aurora-related kinase 1 | 2.29 |
| Aggresomes | gil5729877 | Heat shock 70 kD protein 10 | 6.53 |
| | | (HSV71) | |
| | gil4502549 | Calmodulin 2 (phosphorylase kinase, | 4.84 |
| | | delta) | |
| | gil2119712 | dnaK-type molecular chaperone | 3.21 |
| | | HSPA1L | |

The heat-shock proteins are known to be associated with aggresomes, however, their exact role in their formation is still unclear. The cellular protein aurora-related kinase 1 was present in the nuclear aggresomes, which is involved in chromosome segregation and may act during viral DNA replication. Nucleophosmin was detected in the cytoplasmic aggresomes and not in the nuclear-derived aggresomes. Nucleophosmin is a nucleolar protein that is critical for centresome duplication and genomic stability. Overexpression of nucleophosmin has been noted in a number of malignancies, which may be attributed to its ability to inactivate p53; thus suppressing apoptosis. The inactivation of p53 has been noted in HCMV infected cells although it was thought that this might only be due to the viral immediate early proteins.

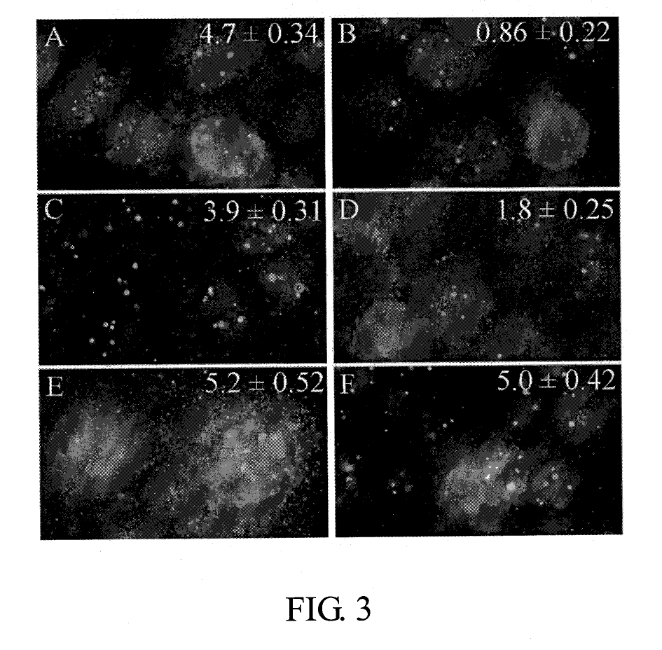

PML bodies are affected by the kinase. Nuclear aggresomes form in a dynamic microtubule dependent process that initiates with fusion of small aggregations located at or near PML bodies. To determine the effect of pUL97 on these structures, this and other proteins were expressed in COST cells and visualized PML bodies with an antibody to SP100, which is a marker of these domains. IE1 has been reported to disperse nuclear structures and reduced their number significantly (FIG. 3B), while the over expression of another viral nuclear protein (ppUL44, ICP36) can not (FIG. 3A). Their number was also unaffected by the expression of pp71, however it colocalized with these structures and is consistent with previous reports. The localization of pp71 near these structures is also consistent with early processes in the formation of nuclear aggresomes and large pp71 aggresomes are frequently observed adjacent to them. Expression of pUL97 also significantly reduced the number of PML bodies (FIG. 3D) in a manner that was kinase dependent since neither the K355M mutant, or pUL97 in the presence of maribavir could affect their numbers (FIG. 3E,F). While this effect was not as robust as the dispersion by IE1, it was both significant and repeatable. These results indicated that the effect of the kinase on PML bodies might be related to its ability to inhibit nuclear aggresome formation.

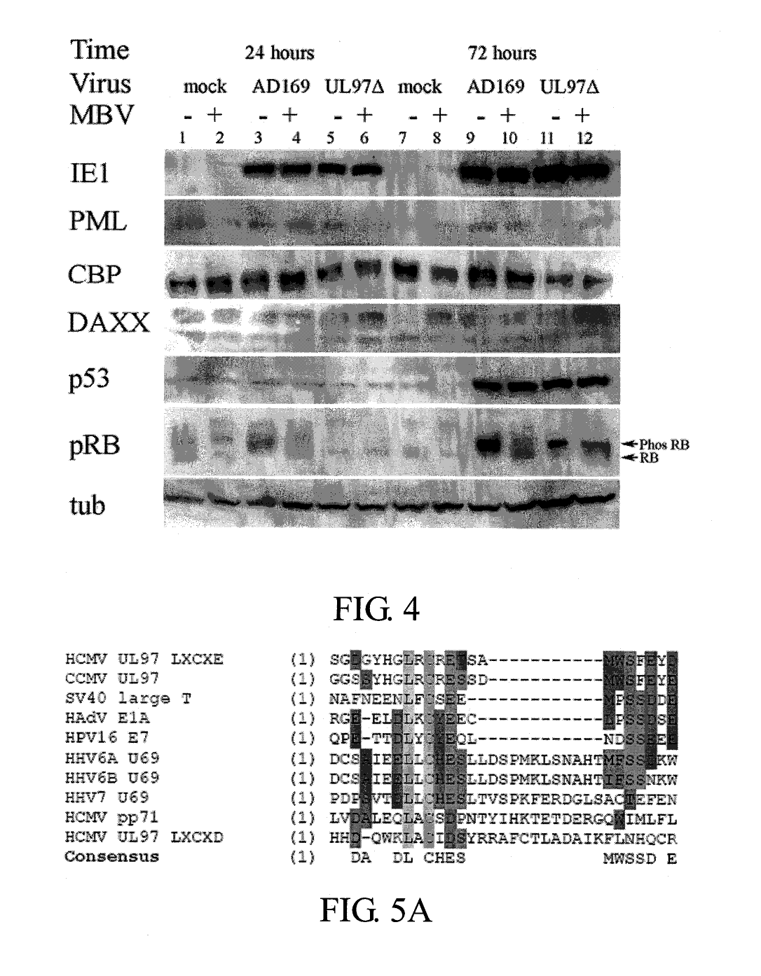

UL97 kinase activity is required for the hyperphosphorylation of Rb. The ability of the kinase to disperse PML bodies suggested that it might be affecting one of the proteins associated with this complex. Proteins in this complex were examined by Western blots using samples derived from HFF cells that were uninfected, infected with AD169 a recombinant virus (RCΔA97) containing a large deletion in UL97 at a multiplicity of infection of 3 PFU/cell. Some of the infections were conducted in the presence of MBV to confirm that the observed changes were due to a deficiency of its kinase activity. No changes were observed in the quantity or sumoylation of PML, CPB or DAXX (FIG. 4). In cells infected with the wt virus, increases in levels of p53 were observed (FIG. 4 lanes 9-12), but this increase was unaffected by the deletion of the UL97 open reading frame or treatment with maribavir. Also consistent with this previous report was an increase of hyperphosphorylated Rb at both 24 and 72 hours following infection (FIG. 4, lanes 3, 9). This did not occur at 24 hours post infection in cells infected with the wt virus that were treated with maribavir (MBV) (FIG. 4 lane 4), or in cells infected with RCΔ97 with or without MBV (FIG. 4 lanes 5, 6). Similarly, at 72 hours after infection, cells infected with the wt virus had high levels of hyperphsophorylated Rb that was significantly reduced with the addition of MBV (FIG. 4 lanes 9, 10) and levels remained low in cells infected with RCΔ97 (FIG. 4 lane 11). Cells infected with the UL97 deletion virus had reduced levels of hyperphosphorylated Rb that was relatively unaffected by the addition of MBV (FIG. 4, compare lane 9 with 11, 12). Taken together, these data confirm that HCMV infection increases levels of hyperphosphorylated Rb and indicate that UL97 kinase activity is required for this effect. Rb interacts directly with PML and has been shown to increase the number of PML bodies. A previous report has shown that pp71 directs the proteasome dependent degradation of hypophosphorylated Rb, however observations presented here are distinct in that the hyperphosphorylation and stabilization of this form of Rb is inefficient in the absence UL97 kinase activity.

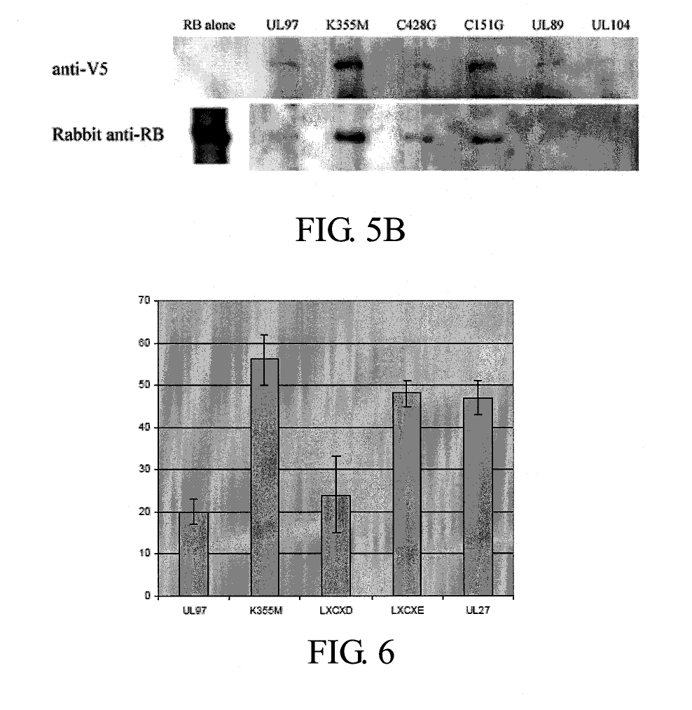

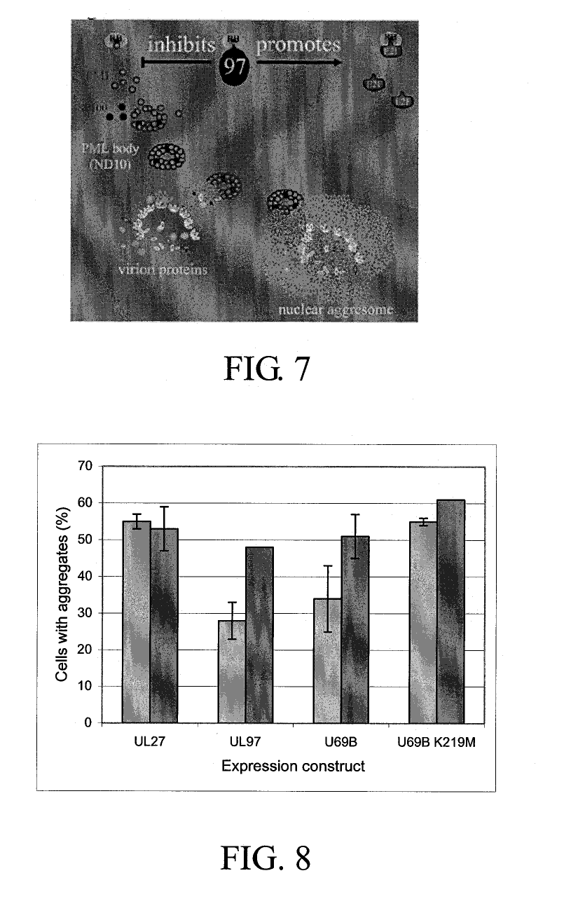

UL97 kinase contains two Rb binding motifs. The amino acid sequence of UL97 kinase was examined for Rb binding motifs. Two binding domains were identified (FIG. 5A). The amino terminus contains a consensus LxCxE (SEQ ID NO:1) motif and an adjacent DSSE (SEQ ID NO:13) motif conserved among proteins that bind Rb including SV40 large T antigen, adenovirus E1A, and HPV E7. The second motif, closer to the carboxyl terminus contains an LxCxD (SEQ ID NO:2) sequence, and is similar to the amino acid sequence required for Rb binding in pp71. The amino terminus of the chimpanzee cytomegalovirus UL97 homolog does not share significant identity with UL97 until the LxCxE (SEQ ID NO:1) motif and DSSE (SEQ ID NO:13) motifs, which are well conserved, as is the LxCxD (SEQ ID NO:2) motif. The UL97 homologs in HHV6-A, HHV6-B, and HHV7 U69, do not share significant amino acid identity with the amino terminus of UL97, yet all retain the LxCxE (SEQ ID NO:1) motif and the DSSE (SEQ ID NO:13) motif is conserved in all the betaherpesviruses except HHV7. The kinase domains of U69 and UL97 are more highly conserved; however the other betaherpesviruses do not retain a conserved the LxCxD (SEQ ID NO:2) motif. The conserved Rb binding motifs in the kinase homologs of the betaherpesviruses are consistent with its affects on Rb and indicate that this function is important in the replication of these viruses.

To explore Rb binding activity further, epitope tagged versions of the kinase were expressed in COST cells and immunopreciptiated with a monoclonal antibody to the V5 epitope. Proteins bound to protein A sepharose beads were incubated with recombinant Rb protein to investigate a potential interaction. Expression of the kinase, as well as the K355M, C151G, C428G mutants were easily detectable using a monoclonal antibody to the epitope tags, as was the expression of two control proteins pUL89 and pUL104 (FIG. 5B). The K355M mutation eliminates kinase activity, while the C151G and C428G mutations disrupt the LxCxE (SEQ ID NO:1) and LxCxD (SEQ ID NO:2) motifs, respectively. Recombinant Rb was specifically precipitated by beads bound to each of the pUL97 proteins, but not to beads bound to either of the negative control proteins. Neither C151G nor C428G appeared to be sufficient to eliminate Rb binding. A similar result was described for BRCA1 where disruption of the LxCxE (SEQ ID NO:1) motif did not disrupt Rb binding, but impaired the inactivation of Rb. Nevertheless, these results indicate that Rb can be co-precipitated by the kinase and is consistent with a direct interaction between the kinase and Rb.

Conserved LxCxE (SEQ ID NO:1) Rb binding motif is required for the inhibition of aggresome formation. Initial studied studies suggested the kinase inhibited the formation of nuclear aggresomes in a process that likely involved PML domains. A subsequent analysis of proteins associated with PML domains identified specific changes in Rb in the presence of kinase activity. Further analysis identified two Rb consensus binding sites in the amino acid sequence of pUL97 and purified Rb binds specifically to the kinase. The kinase might affect Rb directly through the consensus binding domains resulting in an alteration of PML complexes and a failure to form nuclear aggresomes. Each of Rb binding motifs was disrupted with a point mutation to assess their impact on aggresome formation. Each of these plasmids was cotransfected with pp65-EGFP to evaluate their ability to disrupt the formation of nuclear aggresomes induced by this protein. Cotransfection with a plasmid expressing the wt UL97 diminished the formation of nuclear aggresomes, while those expressing the K355M mutant or the UL27 open reading frames had no affect (FIG. 6). Disruption of the LxCxD (SEQ ID NO:2) motif (C428G) did not appear to impair the ability of the kinase to disrupt aggregates to a significant degree. However, a mutation in the LxCxE (SEQ ID NO:1) motif (C151G) eliminated its ability of the kinase to inhibit aggresome formation and results from this plasmid resembled those obtained with the K355M mutant. These results indicate that the kinase affects Rb function directly through the Rb binding motifs. They are also consistent with the kinase affecting nuclear aggresome formation by a mechanism that involves Rb, mediated by PML domains. The essential lysine and the active site (K355M) and the consensus Rb binding motif LxCxE (SEQ ID NO:1) (C151G) were required for the enzyme to inhibit the formation of nuclear aggresomes.

### Example 2

### Maribavir Inhibits the Replication of Human Herpesvirus 6 and the Activity of the U69 Protein Kinase

Maribavir (MBV) was considered a poor inhibitor of HHV-6 replication (Williams-Aziz, S. L., et al. 2005. Comparative activities of lipid esters of cidofovir and cyclic cidofovir against replication of herpesviruses in vitro. Antimicrob Agents Chemother 49:3724-33.). However, as described herein, MBV is a good inhibitor of human cytomegalovirus (HCMV) replication, and the UL97 protein kinase targeted by this drug in HCMV was conserved in HHV-6 (U69). In addition, as shown herein, UL97 inactivated the RB tumor suppressor, which did not occur in the presence of MBV. This result was consistent with the reduced activity of MBV against HCMV in dividing cells where the stimulation of the cell cycle by RB is less important.

The activity of MBV was evaluated against the GS strain of HHV-6A in HSB-2 cells with reduced serum to slow the growth of lymphocytes. Under conditions of reduced serum, MBV exhibited increased activity with EC50 values between 9 and 30 μM, while the EC50 values for the cidofovir controls were unaffected (Table 3).

TABLE 3
Efficacy of maribavir and cidofovir against HHV-6A in HSB-2 cells

EC50 (μM)

| | cidofovir | maribavir |
|---|---|---|
| 10% FBS | 1.4 | >50 |
| 5% FBS | 1.2 | 9 |
| 2% FBS | 1.1 | 30 |

To confirm these results, a surrogate assay was developed for activity of the U69 protein kinase based on its kinase dependent inhibition of nuclear aggregation. This assay is similar to that reported previously for UL97 (Prichard, M. N., et al., 2005. Human cytomegalovirus UL97 Kinase is required for the normal intranuclear distribution of pp65 and virion morphogenesis. J Virol 79:15494-502). As shown in FIG. 8, aggregation of pp65-GFP was inhibited by both the HCMV UL97 kinase and the HHV-6 U69 kinase (light gray bars). Inhibition of aggregation was kinase dependent since the U69B K219M mutation, which eliminates an essential lysine, is incapable of inhibiting aggregation. Treatment of infected cells with 15 μM MBV reduced the ability of UL97 and U69 to inhibit aggregation (FIG. 8, dark gray bars). These results showed that HHV-6B U69 protein kinase is inhibited by MBV. These results also showed that MBV is an inhibitor of HHV-6 replication by targeting UL69.

### Example 3

### Mutation of LxCxE RB Binding Motif and Kinase Motif Impacted RB Stabilization and Phosphorylation

### Materials and Methods

Construction of recombinant viruses. The BAC strain HB5 was mutated using the protocols and plasmids described by Warming et al. (Warming et al., Nucleic Acids Res. 33(4):e36 (2005)). Briefly, PCR was performed using a galK containing plasmid and UL97 galK forward primer 5′-GGC CTT ACG TGC GAC CCG CGT ATG TTC TTG CGC CTT ACG CAT CCC GAG CTC TGC GAC CTG TTG ACA ATT AAT CAT CGG CA-3′ (SEQ ID NO:21) and UL97 galK reverse primer 5′-ATC TTG TGG CAA AAA TCG TCC TCT TTG GGC ACG TAG ACC AGC AGG TAG GAG ATA GAG AGC TCA GCA CTG TCC TGC TCC TT-3′ (SEQ ID NO:22). This PCR product was electroporated into the SW102 recombineering strain containing the pHB5 BAC and plated on selective media containing galactose. The resulting BAC contained a galK insertion at AA 304 and was designated as pMP290. PCR products from plasmids containing K355M, C151G, C428G and C693G point mutations were electroporated into the bacteria containing pMP290, and galK negative BACs were grown on selective media containing deoxygalactose and were designated pMP314, pMP295, pMP312 and pMP316, respectively. Restriction digests of all BACs were conducted to confirm that no large rearrangements had occurred and the UL97 ORFs from each of the resulting BACs was sequenced and confirmed that they contained only the engineered mutations. BACs with the mutations K355M, C151G, C428G, and C693G were rescued and the viruses were designated RC314, RC295, RC312, and RC316, respectively.

Polyacrylamide gels and western blotting. HFF cells were infected at a multiplicity of infection (MOI) of 2 PFU/cell and at 24 and 72 hours following infection they were disrupted in 2× Laemmli buffer (Sigma-Aldrich, St. Louis, Mo.) and separated on 10%, 7.5%, or 5% polyacrylamide gels depending on the size of the protein to be resolved (BioRad, Hercules, Calif.). Separated proteins were transferred to PVDF membranes (Roche Applied Science, Indianapolis, Ind.) in a buffer containing 28 mM Tris, 39 mM glycine, 0.0375% SDS and 20% methanol in a semi dry transfer cell (BioRad, Hercules, Calif.). Membranes were blocked in 1% blocking buffer (Roche Applied Science, Indianapolis, Ind.), incubated with primary antibodies overnight at 4° C. and washed extensively with phosphate buffered saline (PBS) supplemented with 0.02% tween-20. A secondary antibody conjugated to alkaline phosphatase (Southern Biotechnology Associates, Birmingham, Ala.) and CDP* (Roche Applied Science, Indianapolis, Ind.) were used to detect the bound primary antibody. Monoclonal antibodies used in these studies were directed against PML (H-238) (Santa Cruz Biotechnology, Santa Cruz, Calif.), DAXX (Upstate Cell Signaling Solutions, Tamecula, Calif.), p53, RB, β-tubulin, CREB binding protein (CBP) (Chemicon Internationa, Tamecula, Calif.), and both V5 and Xpress (Invitrogen, Carlsbad, Calif.). Rabbit antisera directed against RB phosphorylated on Ser780 (Cell Signaling Technology, Danvers, Mass.) were used in conjunction with Immunopure Goat anti-mouse IgG HRP and Supersignal West Pico Chemiluminescent Substrate (Pierce, Rockford Ill.).

### Results

Four recombinant viruses were constructed in the HB5 BAC strain to assess the role of the conserved RB binding domains on RB stabilization and phosphorylation. These included viruses in which the central cysteines in the LxCxE, LxCxD, and IxCxE domains were mutated to glycines as well as a K355M mutant to confirm that the kinase motif was also required. The entire UL97 ORF was sequenced in each of the viruses to confirm that they contained only the engineered mutations and no large scale rearrangements were detected with a restriction fragment analysis of the BAC DNA. All the viruses appeared to replicate well with the exception of the virus containing the K355M mutation. This virus replicated poorly and its replication kinetics were indistinguishable from those of RCΔ97 in low MOI growth curves. The poor growth phenotype exhibited by the RCΔ97 deletion mutant was due to a deficiency in kinase activity rather than to the disruption of other viral functions.

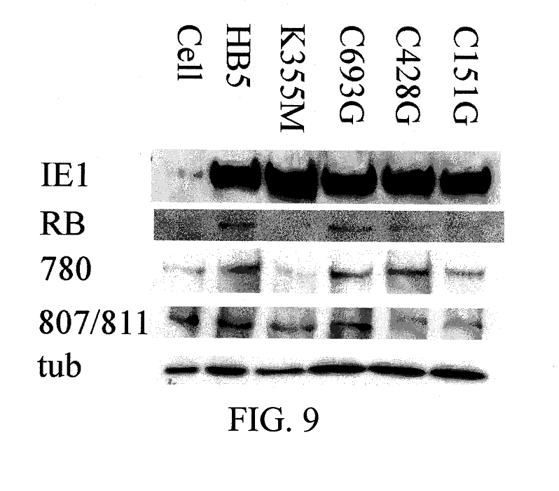

The effect of the four recombinant viruses containing point mutations on RB stabilization and phosphorylation was assessed by western blot using cell lysates harvested at 24 hours after infection (FIG. 9). Cells infected with the wild-type virus stabilized RB. This did not occur when cells were infected with the K355M mutant and confirmed that kinase activity was required for stabilization of RB. The accumulation of RB was reduced in the C151G mutant suggesting this motif was involved with the stabilization of RB. To confirm these data, the phosphorylation of RB was examined using antisera specific for RB phosphorylated on serine 780. The phosphorylation of serine 780 was reduced in the K355M and C151G mutants relative to the wild-type virus and resembled levels seen in uninfected cells. These data suggest that both the conserved LxCxE RB binding motif and the kinase motif were involved in the stabilization and phosphorylation of RB. These results also show the direct interaction and phosphorylation of RB by the UL97 kinase.

1. A method of treating or preventing a proliferative disease in a subject comprising the steps of:
a) selecting a subject with a proliferative disease; and
b) administering an inhibitor of a UL97 or a UL97 homolog to the subject.

2. The method of claim 1, wherein the proliferative disease is selected from the group consisting of heart disease, restenosis, lymphoproliferative disorders, multiple sclerosis, Kaposi's sarcoma, Stevens-Johnson syndrome, post-transplant lymphoproliferative disorder, chronic fatigue syndrome, Burkitt's lymphoma, nasopharyngeal carcinoma, inflammatory disease, organ rejection, transplant arteriosclerosis, myocarditis, retinitis, obliterative bronchiolitis and neoplastic disorders.

3. The method of claim 1, wherein the proliferative disease is not graft versus host disease (GvHD).

4. The method of claim 1, wherein the proliferative disease is associated with a herpes virus.

5. The method of claim 4, wherein the herpes virus is selected from the group consisting of CMV, EBV, HHV-6A, HHV-6B and HHV-7.

6. The method of claim 1, wherein the inhibitor of the UL97 or the UL97 homolog is maribavir.

7. The method of claim 1, wherein the inhibitor of the UL97 or the UL97 homolog is selected from the group consisting of a quinazoline compound, an indolocarbazole, a benzimidazole L-riboside, maribavir (MBV), a derivative of ganciclovir, roscovitine, staurosporine, wortmannin, BIRB796, fasudil, flavopiridol, indurubin, NGIC-I and Go6976

8. The method of claim 1, wherein the inhibitor of the UL97 or the UL97 homolog is a functional nucleic acid.

9. The method of claim 7, wherein the functional nucleic acid is an siRNA, ribozyme or triplex molecule.

10. The method of claim 1, wherein the inhibitor of the UL97 or the UL97 homolog is an inhibitory peptide.

11. The method of claim 10, wherein the inhibitory peptide binds the LxCxE (SEQ ID NO:1) motif of the UL97 or the UL97 homolog.

12. The method of claim 10, wherein the inhibitory peptide binds the LxCxD (SEQ ID NO:2) motif of the UL97 or the UL97 homolog.

13. The method of claim 10, wherein the inhibitory peptide binds the LxCxE (SEQ ID NO:1) and the LxCxD (SEQ ID NO:2) motifs of the UL97 or the UL97 homolog.

14. The method of claim 10, wherein the inhibitory peptide binds the LxCxE (SEQ ID NO:1) motif and the DSSE (SEQ ID NO:13) motif of the UL97 or the UL97 homolog.

15. The method of claim 12, wherein the UL97 homolog is EBV BGLF4, HHV-6A U69, HHV-6B U69 or HHV-7 U69.

16. The method of claim 10, wherein the inhibitory peptide is a dominant negative mutant of a UL97 or a UL97 homolog.

17. The method of claim 1, further comprising administering a therapeutic agent to the subject.

18. The method of claim 17, wherein the therapeutic agent is selected from the group consisting of an anti-cancer compound, anti-opportunistic agents, antibiotics, immunosuppressive agents, anti-virals, anti-inflammatories and immunoglobulins.

19. The method of claim 1, wherein the proliferative disease is cancer.

20. The method of claim 19, wherein the inhibitor of a UL97 or a UL97 homolog is administered locally at or near the site of the tumor.

21. An inhibitory peptide that binds (I) the LxCxE (SEQ ID NO:1) motif of a UL97 or a UL97 homolog, (ii) the LxCxD (SEQ ID NO:2) motif of a UL97 or a UL97 homolog, (iii) the LxCxE (SEQ ID NO:1) and LxCxD (SEQ ID NO:2) motifs of a UL 97 or a UL97 homolog, or (iv) the LxCxE (SEQ ID NO:1) and the DSSE (SEQ ID NO:13) motifs of a UL97 or a UL97 homolog.

22. (canceled)

23. (canceled)

24. (canceled)

25. A medical device comprising an implantable medical device and an inhibitor of a UL97 or a UL97 homolog.

26. The medical device of claim 25, wherein the implantable medical device is selected from the group consisting of a scaffold, a prosthesis, a heart valve, vascular graft, pacemaker, stent, catheter, an intravenous tube and a drug delivery device.

27. The medical device of claim 25, wherein the implantable medical device is administered to a subject with restenosis.

28. The medical device of claim 26, wherein the drug delivery device provides for sustained release of the inhibitor.

29. The method of claim 25, wherein the inhibitor of a UL97 or a UL97 homolog is an inhibitory peptide selected from the group consisting of an inhibitory peptide that binds the LxCxE (SEQ ID NO:1) motif of a UL97 or a UL97 homolog, an inhibitory peptide that binds the LxCxD (SEQ ID NO:2) motif of a UL97 or a UL97 homolog, an inhibitory peptide that binds the LxCxE (SEQ ID NO:1) and LxCxD (SEQ ID NO:2) motifs of a UL97 or a UL97 homolog and an inhibitory peptide that binds the LxCxE (SEQ ID NO:1) and the DSSE (SEQ ID NO:13) motifs of a UL97 or a UL97 homolog.

30. A method of treating or preventing a neoplastic disease in a subject comprising the steps of:
a) selecting a subject with a neoplastic disease; and
b) administering an oncolytic herpesvirus to the subject, wherein the herpesvirus lacks a functional UL97 or a UL97 homolog.

31. The method of claim 30, wherein the herpesvirus is CMV.

32. The method of claim 30, wherein the UL97 comprises the mutation K355M.

33. The method of claim 30, wherein the UL97 or the UL97 homolog comprises a mutation in the LxCxE (SEQ ID NO:1) motif.

34. A method of screening for agents that inhibit the activity of a UL97 or a UL97 homolog comprising the steps of:
(a) providing a cell that expresses a UL97 or a UL97 homolog;
(b) contacting the cell with a candidate agent to be tested; and
(c) determining whether the candidate agent prevents the activation of Rb by the UL97 or the UL97 homolog.

35. A method of screening for agents that inhibit the activity of a UL97 or a UL97 homolog comprising the steps of:
(a) providing a sample comprising a UL97 or a UL97 homolog and Rb;
(b) contacting the sample with a candidate agent to be tested; and
(c) determining whether the candidate agent prevents the activation of Rb by the UL97 or the UL97 homolog.

36. The method of claim 34, wherein the determining step is carried out using a kinase assay or a surrogate assay.

37. The method of claim 35, wherein the determining step is carried out using a kinase assay or surrogate assay.
